(19) United States
(12) Patent Application Publication
(10) Pub. No.: US 2012/0073987 A1
(43) Pub. Date: Mar. 29, 2012

(54) ELECTROCHEMICAL METHOD AND APPARATUS OF IDENTIFYING THE PRESENCE OF A TARGET

(76) Inventors: Heinz-Bernhard Kraatz, Pickering (CA); Kagan Kerman, Toronto (CA)

(21) Appl. No.: 13/376,540

(22) PCT Filed: Jun. 8, 2010
(86) PCT No.: PCT/CA2010/000891

§ 371 Date: Dec. 6, 2011

Related U.S. Application Data
(60) Provisional application No. 61/213,431, filed on Jun. 8, 2009.

(51) Int. Cl. G01N 27/327; B23P 17/00; G01N 33/50

(52) U.S. Cl. 205/782; 204/400; 204/403.01; 204/403.14; 29/401.1

(57) ABSTRACT

An electrochemical method of identifying the presence of a target protein in a sample is provided. The method comprises providing a redox probe modified to include a detector that is suitable to bind to the target protein, and exposing the sample to the detector-modified redox probe. A change in the electrochemical signal produced by the redox probe as compared to a control signal is indicative of the presence of the target protein.

### FIELD OF THE INVENTION

The present invention relates to a novel electrochemical detection method and novel probes for use in electrochemical detection.

### INTRODUCTION

The current treatment for AIDS consists of a highly active anti-retroviral therapy abbreviated as HAART. The anti-retroviral drugs target several essential proteins in HIV and inhibit their functions. First, the fusion inhibitors were designed to stop the entry of the HIV into the host cell. Once the virus enters the cell, the virus RNA is copied into double-stranded cDNA by the viral reverse transcriptase (RT) prior to the integration into the host genome. RT has two essential enzymatic activites: DNA polymerization and cleavage of the RNA strand in RNA/DNA hybrids (RNase H activity). The anti-RT drugs fall into two classes, both of which target the polymerization activity. Most of these drugs are nucleoside analog RT inhibitors (NRTI), which lack the 3′-OH and therefore stop the DNA chain elongation process upon incorporation into the replicating strands. AZT (azidothymidine) was the first FDA-approved NRTI. The non-nucleoside RT inhibitors (NNRTI) bind near the nucleotide-binding site and inhibit the RT function. HIV-1 integrase (IN) catalyzes the insertion of the double-stranded cDNA of the viral genome into the preferred locations within the actively transcribed genes in the infected cell. Mutational analyses of IN established that its function was essential to the viral replication in cell cultures. Besides the drugs against the RT and IN, there is a strong emphasis on the development of drugs against the HIV-1 protease (PR) and other proteins involved in the virus maturation.

Unfortunately, resistance to these drugs can arise rapidly during infection. It was reported that the cross-resistance between the approved anti-RT drugs was often observed and at least 39 of the first 300 codons of HIV-1 RT were associated with drug resistance. The rapid selection of drug-resistant viral strains and the adverse side-effects associated with the long-term exposure to current anti-AIDS drugs necessitate the investigation of alternative drugs. In particular, nucleic acid and peptides that inhibit HIV-1 function by directly binding with the essential enzymes, have emerged as potent inhibitors both in vitro and in cell culture. Their antiviral efficacy is reported to be a function, in part, of the biochemical properties of the peptide-target interaction.

It would be desirable, thus, to provide improved detection methods for use in diagnosis of disease, as well as efficient methods of screening candidate inhibitors, which overcome at least one disadvantage of currently utilized methods.

### SUMMARY OF THE INVENTION

A versatile electrochemical detection method has now been developed which is useful in the diagnosis of disease as well as having utility to screen potential therapeutic compounds.

Thus, in a first aspect of the invention, a redox probe unit suitable for use in the detection of a target is provided comprising a redox probe modified to incorporate a detector-binding moiety adapted to bind a detector having the capacity to specifically interact with the target.

In another aspect of the invention, an electroactive biodetector is provided comprising a redox probe modified to incorporate a detector-binding moiety to which is bound a detector having the capacity to interact with a target.

In another aspect of the invention, a method of making an electroactive biodetector suitable for the detection of a target is provided comprising the steps of:
i) modifying a redox probe to incorporate a detector-binding moiety; and
ii) attaching to the detector-binding moiety a detector which has the capacity to specifically interact with the target.

In another aspect of the invention, an electrochemical method of detecting a target in a sample is provided. The method comprises exposing the sample to a redox probe modified to include a detector that is suitable to bind to the target, wherein the detector is linked to the probe via a detector binding moiety, and measuring the electrochemical signal produced by the redox probe, wherein a change in the electrochemical signal of the probe as compared with control is indicative of the presence of the target in the sample.

In a further aspect of the invention, an electroactive biodetector unit adapted to detect multiple targets in a sample is provided. The electroactive biodetector unit comprises multiple biodetectors each of which is adapted to detect a different target.

These and other aspects of the invention are described by reference to the detailed description the following Figures.

### BRIEF DESCRIPTION OF THE FIGURES

FIG. 1 is a schematic illustrating the reaction of a labelled ferrocene-modified electrode with a target protein;

FIG. 2 illustrates representative cyclic voltammograms of Au microelectrodes at different stages during the preparation of peptide-ferrocene modification;

FIG. 3 illustrates cyclic voltammograms (A), linear plots of formal potential (B) and current density (C) of HIV-1RT-modified Au microelectrodes in the presence various concentrations of HIV-1 RT;

FIG. 4 illustrates cyclic voltammograms (A), linear plots of formal potential (B) and current density (C) of HIV-1 IN-modified Au microelectrodes in the presence various concentrations of HIV-1 IN;

FIG. 5 graphically illustrates the effect of pH on the interaction of HIV-1 peptides and their respective target proteins;

FIG. 6 graphically illustrates the effect of NaCl concentration on the interaction of HIV-1 peptides and their respective target proteins;

FIG. 7 graphically illustrates the specificity of the interaction of HIV-1 peptides and their respective target proteins;

FIG. 8 is a schematic illustrating the use of labelled and unlabeled redox probes in an electrochemical method;

FIG. 9 illustrates cyclic voltammograms (A) and Faradic impedance spectra (B) of an unlabeled probe;

FIG. 10 illustrates a Nyquist plot (−Zᵢₘ vs Zᵣₑ) of impedance spectra obtained at different RT concentrations using an unlabeled probe (A) and a calibration plot of R_{CT} and R_{X} vs RT concentration;

FIG. 11 illustrates cyclic voltammograms (A), square wave voltammograms (B) and differential pulse voltammograms (C) using a labeled RT probe;

FIG. 12 illustrates cyclic voltammograms (A) of a labelled probe at different scan rates and a plot of the linear relationship between the scan rate and the anodic and cathodic peak currents for the bound film (B); and

FIG. 13 illustrates a Nyquist plot (−Zᵢₘ vs Zᵣₑ) of impedance spectra obtained at different RT concentrations (A) and a calibration plot of R_{CT} and R_{X} vs RT concentration (B).

### DETAILED DESCRIPTION OF THE INVENTION

An electroactive biodetector that is useful to detect a target compound is provided. The biodetector comprises a redox probe modified to incorporate a detector-binding moiety. The biodetector is prepared by modifying the redox probe to incorporate a detector-binding moiety and attaching to the detector-binding moiety a detector that has the capacity to specifically interact with the target compound.

The “redox probe” may be any electroactive material that undergoes a reversible redox reaction on the application of a potential and which is suitable for use with biomolecules such as proteins and nucleic acids. In one embodiment, the redox probe comprises an electrode. The electrode may be formed of any electrically conducting material, including for example, gold, silver, copper, aluminum, indium tin oxide (ITO) and the like. In another embodiment, the redox probe comprises an electrode labelled with a molecule that undergoes a reversible one-electron redox process such as a metallocene, quinones e.g. quinone/hydroxyquinone is one redox couple that is pH sensitive, anthraquinone, [Ru(NH3)6]2+/3+, and [Ru(bipy)3]2+/3+. A molecule that undergoes a reversible one-electron redox process, such as a metallocene, may be immobilized on the surface of the electrode. The term “metallocene” is used herein to encompass metallocene compounds and derivatives thereof, including ferrocene, cobaltocene, and derivatives thereof.

To prepare the biodetector, the redox probe is modified to incorporate a detector-binding moiety at its surface to form a redox probe unit. The nature of the modification will depend on the detector-binding moiety to be incorporated onto the redox probe but will generally involve techniques well-established in the art. Generally, the term “a detector-binding moiety” refers to a moiety reactive to form a linkage with a detector, a compound suitable to specifically interact with a specific target, such as a target protein, in an electrolyte solution. For protein or peptide detectors, the detector-binding moiety may be a reactive carboxyl or amino group which is suitable to form an amide linkage with the detector. The detector-binding moiety may also be a moiety suitable to form a linkage with a side group of the detector protein or peptide (e.g. side-chain groups such as the imidazole ring in histidine residues of proteins). In order to maintain the function of the detector to bind to a specific target, the detector-binding moiety should not interfere, e.g. form a linkage, with the site on the detector required to interact with the target protein. For oligonucleotide detectors, the detector-binding moiety may also be a reactive carboxyl or amino group, or may additionally be hydroxide, sulfhydryl, active ester or halide.

The redox probe unit advantageously provides a unit that is adaptable with respect to the target that it may be used to detect and may be used to detect a range of different targets depending on the detector linked thereto. Thus, a given redox unit may be utilized to detect a variety of targets by simply changing the detector linked to the redox unit.

A selected detector may be linked to the redox probe by attachment to the detector-binding moiety using methods appropriate for the particular detector and binding moiety. For example, linkage of a peptide detector to reactive carboxyl or amino group will employ methods suitable for this reaction to occur. Detectors for use in the preparation of the present electroactive biodetector are selected based on their specificity for the target. Thus, appropriate detectors include ligands, such as peptide or nucleic acid ligands, of the target. As used herein, the term “target” is meant to encompass any entity detectable through ligand binding, including proteins, such as viral and non-viral proteins, glycoproteins such as antibodies, hormones and antigens such as prostate-specific antigen (PSA). For the detection of HIV-1 in a given sample, the detector may be selected from ligands for HIV-1 enzymes, including HIV-1 reverse transcriptase, HIV-1 integrase and HIV-1 protease. The present biodetector may be used for the detection/diagnosis of other viral infections, by utilizing a ligand as the detector which specifically binds to a target viral protein.

An electrochemical method of detecting a target in a sample is also provided in another aspect of the invention. The method comprises exposing a sample to a redox probe modified to include a detector that will specifically bind to the target and applying a potential to the redox probe. A change in the electrochemical signal produced by the redox probe in comparison to the signal produced in the presence of a control solution, e.g. a sample that does not contain target, is indicative of the presence of the target protein in the sample. The term “sample” is used herein to encompass samples that may contain a target protein, and include biological samples such as blood, plasma, urine, sweat, tears and saliva. As one of skill in the art will appreciate, the amount of target may also be quantified using the present detection method by comparison to quantified standards as exemplified herein.

A change in the electrochemical signal may be measured using any appropriate technique, including cyclic voltammetry, differential pulse voltammetry, square wave voltammetry, alternating current voltammetry, and impedance spectroscopy. Changes in the electrochemical signal such as an anodic shift in the formal potential, or a decrease in signal strength (e.g. current density) are both indicators of the presence of a target in a sample.

While not wishing to be bound by any particular theory, the detection principal of the present method appears to be based on modulation of the electrochemical signal as a result of steric hindrance. When the detector of the redox probe is not bound with target, the electrolyte ions facilitate electron transfer to electrode surface. Upon the incubation of the redox probe with target, and binding of target to the probe, the detector is enveloped in a target environment, e.g. protein environment where the target is a protein, which hinders electron transfer at the electrode surface, e.g. communication of the metallocene with the electrode. Thus, the binding reaction results in modulation of the electrochemical signals that may be recorded as a shift in the peak potential or a decrease in the intensity of current.

Thus, the present electrochemical method may be used for the detection/diagnosis of disease, such as viral infection, by utilizing a ligand as the detector which specifically binds to a target viral protein or other target proteins indicative of disease, e.g. antibodies.

The present method may also be utilized to screen for candidate therapeutic compounds that target a particular protein. In this regard, a target protein ligand may be linked to the redox probe as the detector and then exposed to a solution containing the target protein and a candidate therapeutic compound. A change in the electrochemical signal produced by the redox probe on application of a potential to the solution in comparison to the signal produced in the presence of a control solution, e.g. a sample that does not contain a candidate therapeutic, may indicate that the candidate is a potential therapeutic compound, e.g. a potential modulator or inhibitor of the target protein.

In a further aspect of the invention, an electroactive biodetector unit adapted to detect multiple target proteins is provided. The electroactive biodetector unit comprises multiple biodetectors each of which is adapted to detect a different target proteins. In this regard, the biodetector unit may be adapted to detect different targets that may be present in a single sample, and may be targets of a single organism, e.g. a single virus such as HIV-1, such as HIV-1 reverse transcriptase, HIV-1 integrase and HIV-1 protease, or target proteins of multiple organisms, e.g. HIV-1, Hepatitis C virus, Cytomegalovirus or target proteins of different types, e.g. antibodies to a series of antigens of interest, and nucleic acids, e.g. DNA and RNA-based aptamers and hybridization oligonucleotides with sequences related to specific diseases.

Embodiments of the invention are described by reference to the following specific example which is not to be construed as limiting.

### EXAMPLE 1

### Electrochemical Detection of Proteins Using a Labeled Probe

### Reagents and Apparatus:

HIV-1 reverse transcriptase (200 U, T3610Y, RT) was purchased from GE Healthcare (Quebec, Canada). HIV integrase (100 μg, HIV-122, IN) was purchased from ProspecBio (USA). Bovine serum albumin (BSA), HIV-1 protease (PR) and its inhibitor peptide pepstatin (VVStaASta) were purchased from Sigma-Aldrich (Canada). The peptide ligands for RT (VEAIIRILQQLLFIH) (SEQ ID No: 1) and IN (YQLLIRMIYKNI) (SEQ ID No: 2) were purchased from BioBasic (ON, Canada).

The synthesis of the thioctic acid-modified Ferrocene (Thc-Fc) for surface modification was prepared as described by Mahmoud et al. (Chem. Eur. J, 2007, 13, 5885-5895), the relevant contents of which are incorporated herein by reference. Gold microelectrodes (25 μm i.d.) were prepared in the laboratory using well-established methodology, e.g. Mahmoud et al. 2007. The covalent agents, 1-ethyl-3-(3-dimethylaminopropyl)carbodiimide HCl (EDC) and N-hydroxysuccimide acid (NHS) were purchased from Pierce (USA). All the other chemicals were obtained from Merck & Co. Inc. and used as received.

### Preparation of Peptide-Modified Gold Electrodes:

Au microelectrodes were incubated in 1 mM ethanolic solution of Thc-Fc overnight (˜15 h). The electrodes were rinsed with ethanol and Millipore water (18.2 MΩ.cm). This layer was terminated by the amino-reactive carboxyl moieties that allowed the attachment of the peptides. The carboxyl groups were activated with 2 mM EDC and 5 mM NHS in 50 mM phosphate buffer solution (pH 7.4) for 2 h. The N-terminus of the peptides was attached to the activated carboxyl groups of Thc-Fc to give the final electrode interface. The peptides (1 mM) were incubated with the activated electrodes in 50 mM phosphate buffer solution (pH 7.4) for 2 h on a block-shaker at room temperature. Thus, the peptides were conjugated with the Fc compounds on the surface. The remaining active-esters were quenched by incubating the peptide-modified electrodes in 100 mM ethanolamine solution for 1 h on a block-shaker at room temperature. The peptide-modified electrodes were then incubated in 1 mM hexanethiol solution for 5 min to produce a diluted film and to back-fill the empty spots on the electrode surface. Finally, the electrodes were rinsed with ethanol and Millipore water to give the peptide-modified sensor surfaces.

Incubation with HIV Enzymes:

The stock solutions of the enzymes RT and IN (200 nM) were prepared using their assay buffers containing 25 mM MOPS (pH 7.2) with 100 mM NaCl and 7.5 mM MnCl2 and 20 mM HEPES (pH 7.5) with 10 mM NaCl and 7.5 mM MnCl2, respectively. Several dilutions of the RT and IN stock solutions were prepared using their respective assay buffers. The electrodes were incubated with these RT and IN samples for 1 h and then rinsed with Millipore water.

### Electrochemical Measurements:

Electrochemistry was carried out at room temperature using an electrochemical cell enclosed in a grounded Faraday cage. A Luggin capillary was used to make electrochemical connection to Ag/AgCl reference electrode. Pt wire was used as the counter electrode. Electrochemical measurements were performed in 2 M NaClO4 as the supporting electrolyte. Error bars in the plots indicate the standard deviation of the electrochemical responses from five repetitive measurements (n=5). Cyclic voltammetry experiments were performed at varying scan rates using a CHInstruments 660 potentiostat/galvanostat.

### Results and Discussion

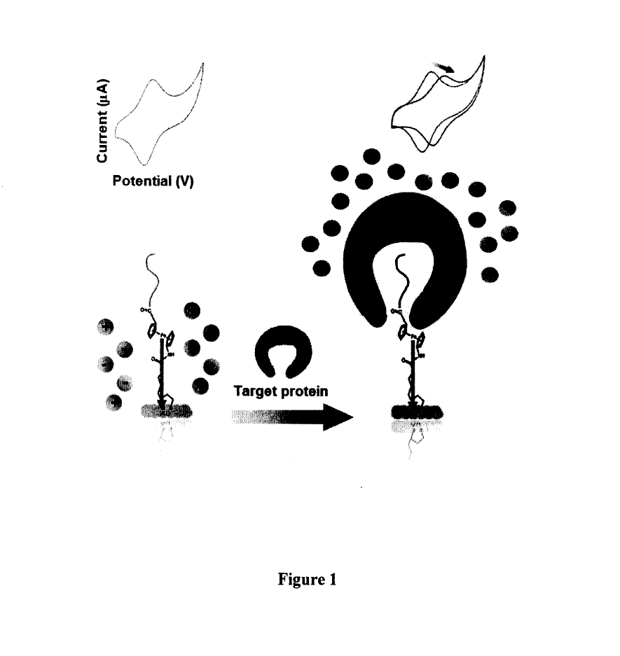

The modulation of electrochemical signals is used for the detection of the interaction between HIV enzymes and their inhibitory peptides, making use of a ferrocene (Fc)-modified electrode. The close proximity of the electro-active Fc moiety to the electrode surface created a sensitive layer towards further modifications above the Fc layer. When the binding reactions between the peptides and the HIV enzymes took place, the Fc layer was immersed in a protein environment, which affected its redox activity by hindering the accessibility of the electrolyte ions to the electrode surface. Thus, modulation of the electrochemical signal was recorded as changes in the intensity of the Fc current signals and the shifts in the anodic peak potential. FIG. 1 is a schematic illustrating the reaction of the ferrocene-modified electrode with a target protein and the effect on anodic peak potential.

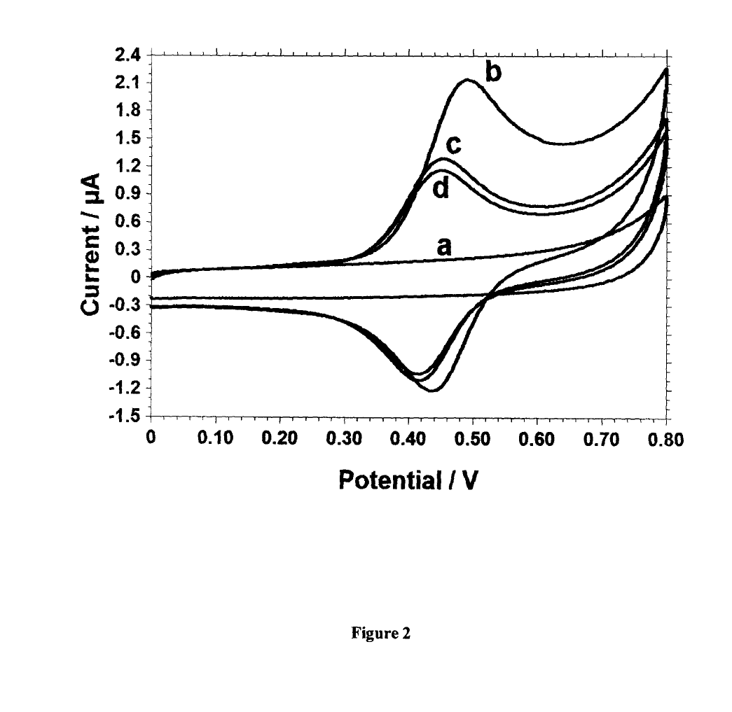

The peptides were immobilized on Au microelectrodes by immersing them in a 1 mM solution of the peptide in PBS. Cyclic voltammograms (CVs) were recorded in aqueous solutions of 2 M NaClO₄ at a scan rate of 100 mV s⁻¹. The peak current from the CVs increased linearly with scan rate, as was expected for an adsorbed film. FIG. 2 shows the representative CVs of Au microelectrodes during the modification stages as follows: (a) bare Au microelectrode in blank 2 M NaClO₄ solution, (b) after the immobilization of Thc-Fc molecules on the surface, (c) after the attachment of peptide-IN with the surface-anchored Thc-Fc molecules, (d) after the quenching of active ester groups using 100 mM ethanolamine and the backfilling of empty spots on the surface using 1 mM hexanethiol. The CVs display a decrease in the current response as the peptides were attached on the Thc-Fc-modified electrode. A slight decrease in the CV current responses was observed and the signals were stabilized after the quenching of the remaining active-esters using 100 mM ethanolamine and the back-filling of the non-modified surfaces using 1 mM hexanethiol. Table 1 displays the statistical evaluation of the surface-modification stages.

TABLE 1
Summary of the electrochemical properties of films of Thc-Fc-peptide
conjugates on Au microelectrodes.*

| Layers | E⁰ (mV) | ΔE (mV) | Γ (mol · cm⁻²) |
|---|---|---|---|
| Thc-Fc | 480 (±25) | 89 (±10) | 3.5 × 10⁻¹¹ |
| Peptide | 455 (±15) | 67 (±15) | 1.7 × 10⁻¹¹ |
| Ethanolamine | 420 (±10) | 40 (±15) | 1.1 × 10⁻¹¹ |
| Hexanethiol | 420 (±10) | 45 (±15) | 0.9 × 10⁻¹¹ |

*Surface concentrations Γ are calculated in mol · cm⁻² with supporting electrolyte 2M NaClO₄ at pH 7, Ag/AgCl reference electrode, Au working microelectrode and Pt wire counter electrode.

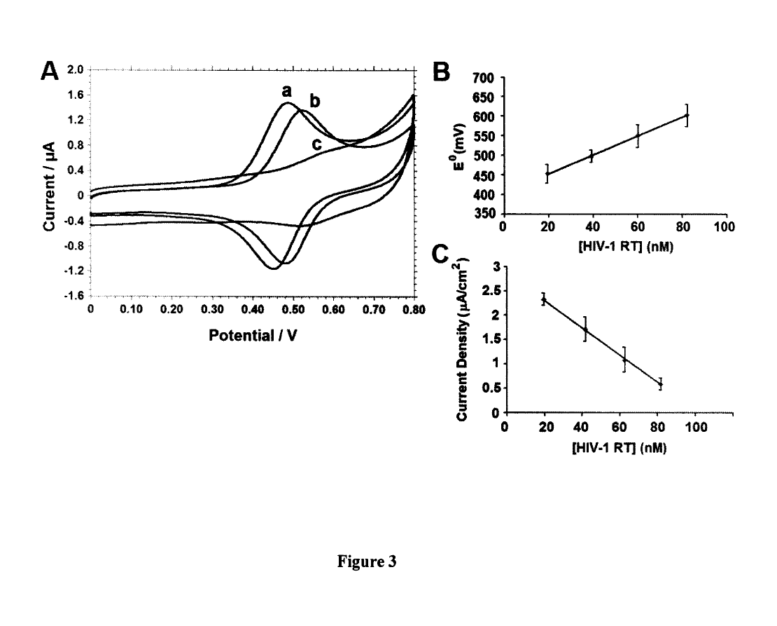

Moreover, the integration of the Faradaic peak currents of the CV curves allowed evaluation of the surface concentration of the peptide film covalently linked to the surface. FIG. 3A shows the cyclic voltammograms of Au microelectrodes modified with peptide-RT (VEAIIRILQQLLFIH) in the presence of (a) no HIV-1 RT, (b) 50 nM HIV-1 RT and (c) 80 nM HIV-1 RT at a scan rate of 100 mV s⁻¹ in 2 M NaClO₄. Plots for the linear relationship of (B) the formal potential and (C) the current density with HIV-1 RT concentration (n=3). HIV-1 RT assay buffer included 25 mM MOPS (pH 7.2) with 100 mM NaCl and 7.5 mM MnCl₂.

As the enzyme concentration increased, the formal potential for the surface-bound peptide shifted to higher potentials, indicating that the oxidation of the Fc group was becoming increasingly more difficult. This interaction was plotted with E° (FIG. 3B) and current density (FIG. 3C) as a function of HIV-1 RT concentration. A linear relationship was observed for RT concentration up to 80 nM. Afterwards, the potential reached a steady state indicating potentially the saturation of the surface with HIV-1 RT. The detection limit was 20 nM estimated from 3(S_{b}/m), where S_{b} is the standard deviation of the measurement signal for the blank and m the slope of the concentration curve in the linear region. Apart from the anodic shift in the formal potential, a linear decrease in the current density was observed (FIG. 3C) from 20 to 80 nM of RT.

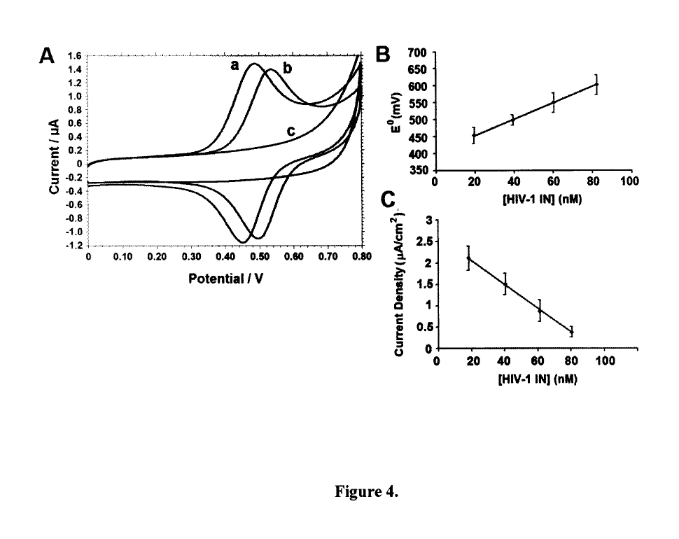

Similar plots were obtained for the interaction of HIV-1 IN with peptide-IN-modified Au microelectrodes. The microelectrodes were incubated with the assay buffer containing various concentrations of IN. FIG. 4A illustrates cyclic voltammograms of Au microelectrodes modified with peptide-IN (YQLLIRMIYKNI) (SEQ ID No. 2) in the absence of HIV-1 IN (a), and in the presence of 40 nM IN (b) and 100 nM IN (c) obtained at a scan rate of 100 mV s⁻¹ in 2 M NaClO₄. Plots for the linear relationship of the formal potential (FIG. 4B) and the current density (FIG. 4C) with HIV-1 IN concentration (n=3) were also obtained. HIV-1 IN assay buffer included 20 mM HEPES (pH 7.5) with 10 mM NaCl and 7.5 mM MnCl₂. The detection limit for HIV-1 IN was also determined as 20 nM with a linear relationship from 20 to 80 nM. As the peptide film became more crowded upon the specific binding of the HIV-1 IN with the IN peptide, the ability of the supporting electrolyte ions to penetrate the film decreased, resulting in the changes in the electrochemical behavior of the Fc.

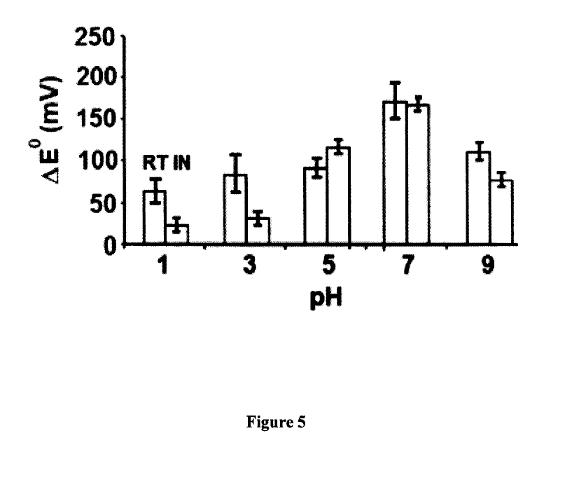

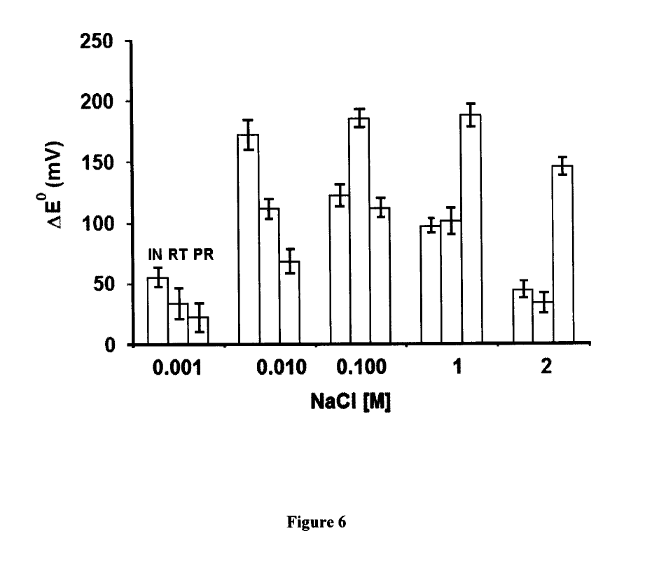

Incubation of electrodes modified with peptides RT and IN with solutions of HIV-1 RT and IN, respectively, was conducted in solutions of varying pH. As shown in FIG. 5, the binding process was affected by the changes in the pH of the solution. Interactions of RT and IN with HIV-1 RT and IN, respectively, were favoured at neutral pH which facilitated the binding of the enzymes to their respective peptides. Under acidic conditions, negligible interactions with the surface-bound peptides were observed. The effect of NaCl concentration was similarly determined (FIG. 6). Electrodes with peptides RT, IN and PR were prepared (n=3) and each electrode was interacted with 100 nM solutions of the enzymes, HIV-1 RT, IN and PR (Pepstatin), respectively. Significant dependence on the NaCl concentration was observed for each enzyme. None of the enzymes showed extensive binding activity at 1 mM NaCl condition. The binding of HIV-1 IN was observed to increase at 10 mM NaCl condition, whereas HIV-1 RT displayed the highest shifts in its peak potential at 100 nM NaCl. However, HIV-1 PR required a high ionic strength at 1 M NaCl for its optimum binding efficiency. These measurements indicated that solution conditions such as pH and ionic strength should be adjusted according to the targeted enzyme for the optimum performance of the biosensor.

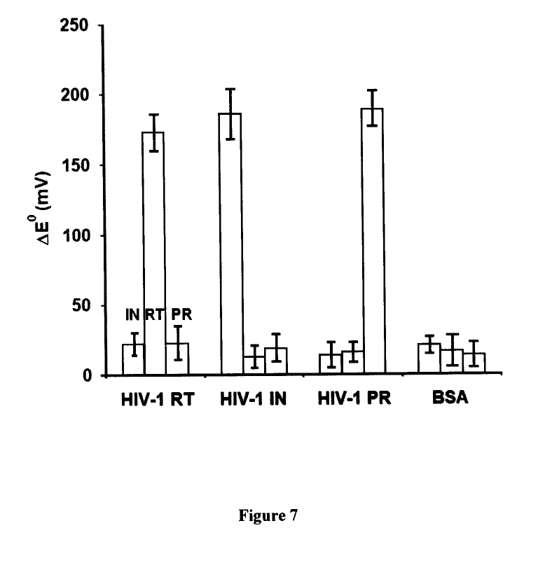

Control experiments were performed using peptide-modified electrodes in order to test non-specific binding of various proteins on the surface as shown in FIG. 7. Electrodes (n=3) were prepared with peptides RT, IN and PR. These electrodes were exposed to the 100 nM solutions of RT, IN, PR and BSA to determine the selectivity of the peptides towards their target proteins. As expected, the peptide-RT responded to the exposure of HIV-1 RT, whereas the peptide-IN responded to the exposure of HIV-1 IN. However, none of the peptide-modified surfaces reacted with the BSA to cause significant changes in the electrochemical signal. Moreover, the Fe-modified electrodes were exposed to 100 mM ethanolamine to create peptide-free surfaces. Measurements upon incubation of these control electrodes with the HIV enzymes and BSA did not cause significant changes in the electrochemical signals of Fc. These measurements indicated that the non-specific adsorption of proteins on the surfaces was negligible.

### Conclusions

An electrochemical method of detecting various clinically important proteins that have no redox-active centers is provided. The approach is versatile and useful to detect numerous proteins by altering the recognition site of the electrode. The method may also be adapted for the detection of multiple proteins in a microarray format and high throughput screening of candidate inhibitors (peptides or nucleic acids) of these enzymes.

### EXAMPLE 2

### Electrochemical Detection of Proteins Using an Unlabeled Probe

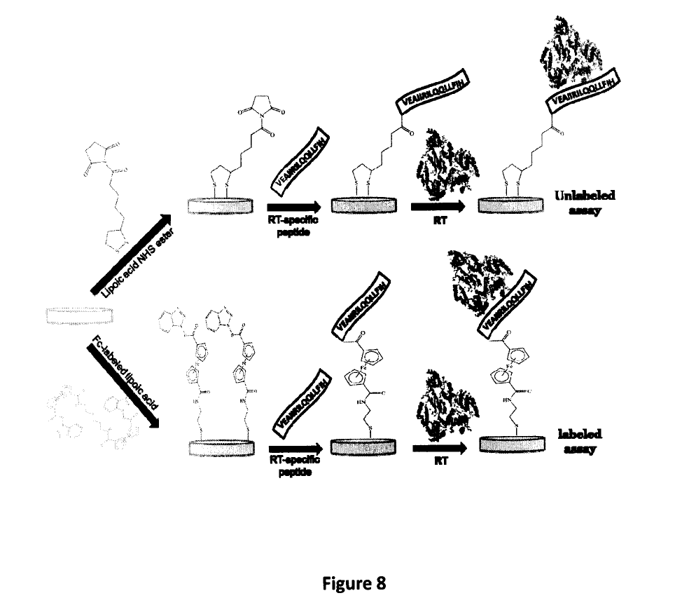

A comparison experiment was conducted as follows. The present electrochemical detection method was conducted using an unlabeled electrode and an electrode labelled with a molecule that undergoes a reversible one-electron redox process (e.g. a metallocene such as ferrocene (Fc)). FIG. 8 illustrates the labelled and unlabeled versions of the present detection method.

### Materials and Reagents

The working gold electrodes, 99.99% (w/w) polycrystalline with a 1.6-mm diameter and 0.02 cm² geometrical area, were purchased from CH Instrument Inc. and cleaned prior to use. Sputtering gold electrodes were prepared by evaporating 200 nm of gold on a silicon wafer with 2 nm of chromium as the adhesive layer. HIV-1 reverse transcriptase enzyme was purchased from Applied Biosystems (Streetsville, ON, Canada). The RT-specific peptide (VEAIIRILQQLLFIH) was purchased from Bio Basic Inc. (Markham, ON, Canada). NaClO₄, K₄[Fe(CN)₆], ethanolamine, and hexanethiol were purchased from Aldrich and used without further purification. Deionized water (18.2 MΩ.cm resistivity) from a Millipore Milli-Q system was used throughout this work. Ethanol used throughout the work was freshly distilled prior to use. Unless indicated, all measurements were carried out at room temperature (22 ° C.±2).

### Synthesis of the Ferrocene-Labeled Lipoic Acid Derivative

Synthesis of [CH₃OOC-Fc-CONHCH₂CH₂S]₂-1′-methoxycarbonylferrocene-1-carboxylic acid (1 equiv.), HBTU (1.2 equiv.), Et₃N (2 equiv.) were stirred in 100 mL DCM for 2 hours. After which cystamine (0.75 equiv.) was added and the reaction mixture was stirred for 72 hours. 150 mL DCM was added and the reaction mixture was washed with two 150 mL portions of H₂O and the organic layer collected and dried over Na₂SO₄. The crude mixture was filtered and the solvent removed en vacuo to leave an orange oil. The oil was purified by flash chromatography on silica using diethyl ether as the eluent. The product was in the third fraction and was concentrated in vacuo. The product could be isolated as feathery crystal upon addition of hexanes. Yield 62%.

¹H NMR (400 MHz, CDCl₃) δ 6.88 (s, 2H), 4.83-4.75 (m, 4H), 4.75-4.66 (m, 4H), 4.51-4.42 (m, 4H), 4.40-4.33 (m, 4H), 3.80 (s, 6H), 3.73 (q, J=6.4, 4H), 3.00 (t, J=6.5, 4H).

¹³C NMR (101 MHz, cdcl₃) δ 172.0, 169.8, 78.2, 73.1, 72.4, 72.0, 71.9, 70.4, 52.2, 39.1, 38.3. m/z : 715.0312 (Na⁺): 692.0401 (calc.)

Synthesis: [HOOC-Fc-CONHCH₂CH₂S]₂—[CH₃OOC-Fc-CONHCH₂CH₂S]₂ (1 equiv.), LiOH (5 equiv.), H₂O (1 mL) and THF (15 mL) were stirred at room temperature for 48 hours. The THF was removed in vacou and 10 mL of 0.1 M NaOH was added. The aqueous solution was poured into 1M HCl to precipitate the product which was collected in a sintered glass crucible. Yield 21%.

¹H NMR (400 MHz, acetone) δ 7.79 (s, 2H), 4.93 (t, J=1.9, 4H), 4.82 (t, J=1.9, 4H), 4.52 (t, J=1.9, 4H), 4.44 (t, J=1.9, 4H), 3.77-3.64 (m, 4H), 3.14-3.00 (m, 4H).

¹³C NMR (101 MHz, acetone) δ 172.1, 169.9, 79.1, 74.1, 73.7, 72.8, 71.0, 40.0, 38.6. m/z=686.9988 (Na⁺); 664.0088(calc.).

Synthesis: [C₆H₄N₃OOC-Fc-CONHCH₂CH₂S]₂—[HOOC-Fc-CONHCH₂CH₂S]₂ (1 equiv.), HOBt (1.5 equiv.), and EDC (1.5 equiv.) were stirred for 2 hours in 15 mL of DCM at room temperature. The reaction mixture was diluted with DCM and wash with saturated sodium bicarbonate solution, 10% citric acid solution, and H₂O. The organic phase was dried over Na₂SO₄, filtered and the solvent removed in vacuo. The crude product was purified using flash chromatography on silica using ethyl acetate as the eluent. The product was isolated as an orange solid. Yield 20%.

¹H NMR (400 MHz, cdcl₃) δ 8.05 (d, J=8.4, 2H), 7.56-7.47 (m, 4H), 7.41 (ddd, J=8.3, 6.5, 1.5, 2H), 7.15 (t, J=5.8, 1H), 5.07-5.02 (m, 4H), 5.02-4.97 (m, 4H), 4.76-4.71 (m, 5H), 4.57-4.52 (m, 4H), 3.66-3.56 (m, 4H), 2.83 (t, J=6.6, 4H).

¹³C NMR (101 MHz, cdcl₃) δ 168.9, 168.0, 159.6, 143.8, 129.3, 129.2 125.3, 120.7, 108.8, 78.9, 75.4, 73.0, 72.9, 71.45, 64.8, 39.3, 38.1. m/z=921.0619 (Na⁺); 898.0742 (calc.)

### Preparation of the Peptide-Modified Gold Electrodes

Prior to experiment, the gold electrode was polished with 0.3 and 0.05 μm alumina slurry, cleaned in 0.5 M KOH, and then washed thoroughly with Millipore water. Next, the electrode surface was cleaned by electrochemical sweeping in 0.5 M H₂SO₄ within the potential range of 0-1.5 V until a stable gold oxidation peak at 1.1 V vs Ag/AgCl was obtained. The real electrode surface area and roughness factors were obtained by integrating the gold oxide reduction peak and were found as and, respectively [42]. Consequently, the gold surface was scanned cyclically between −3 and −0.2 V until a stable baseline at 0 V is formed, indicating the reduction of any gold oxide. Finally, the gold electrode was washed with Millipore water, dried, soaked in an ultrasonic bath with ethanol for 5 min, and then dried with N₂. The self-assembling of the lipoic acid NHS ester on the gold surface was performed by dipping the electrode into a dry acetonitrile solution containing 2 mM of the active ester for 24 h, at room temperature. On the other hand, self-assembling of the Fc-labeled lipoic acid derivative on the gold surface was carried out using 2 mM of the Fc-derivative in ethanol under the same conditions. Afterwards, the electrode was incubated with 1 mM of the RT-specific peptide in 10 mM sodium phosphate buffer (pH 7) overnight at 4° C. The remaining active esters were quenched by incubating the peptide-modified electrode in 100 mM ethanolamine solution in ethanol for 1 h at room temperature. Subsequently, the peptide-modified electrode was incubated with 1 mM hexanethiol solution in ethanol for 10 min to produce a diluted film and to back-fill the empty spots of the electrode surface. Finally, the electrode was rinsed with ethanol and Millipore water to give the peptide-modified sensor surface.

Incubation with HIV RT

Incubation with HIV RT enzyme was conducted as described in Example 1.

### Electrochemical Measurements

All electrochemical studies, including cyclic voltammetric (CV), square wave voltammetric (SWV), differential pulse voltammetric (DPV) measurements, and electrochemical impedance spectroscopy (EIS) were performed with an electrochemical analyzer (CH Instruments, Austin, Tex., USA), connected to a personal computer. All measurements were carried out at room temperature in an enclosed and grounded Faraday cage. A conventional three-electrode system was used: a peptide-modified gold electrode as working electrode, a platinum wire as a counter electrode, and a Ag/AgCl/3 M NaCl as a reference electrode. The reference electrode was always isolated from the cell by a miniature salt bridge (agar plus KNO₃) to avoid the leakage of the CF ions from the reference electrode to the measurement system. All potentials were reported with respect to the Ag/AgCl/3 M NaCl reference electrode. The open-circuit or rest potential of the system was measured prior to all electrochemical experiments to prevent sudden potential-related changes in the SAM. All electrochemical experiments were started from the rest-potential. Electrochemical measurements of the unlabeled biosensor, with the RT-specific peptide attached to the gold surface via the lipic acid NHS ester, were performed in 10 mM sodium phosphate buffer (pH 7), containing 100 mM NaClO₄ and 5mM K₄Fe(CN)₆, at the formal potential of the Fe(CN)₆⁻⁴ probe (250 mV). On the other hand, measurements of the labeled biosensor, with the peptide attached to the gold surface via the Fc-labeled lipoic acid derivative, were carried out in 10 mM sodium phosphate buffer (pH 7), at the formal potential of the disubstituted ferrocene derivative (750 mV). All CV experiments were performed at a scan rate of 100 mV s⁻¹ in the potential range of 0-600 mV for the unlabeled biosensor and 200-1100 mV for the labeled counterpart. Both SWV and DPV experiments for the labeled biosensor were carried out in the range from 500 to 1000 mV with a step potential of 4 mV, amplitude of 25 mV, and a frequency of 15 Hz for SWV. All EIS experiments were conducted in the frequency range of 100 kHz to 0.1 Hz with AC amplitude of 5 mV. The measured EIS spectra were analyzed with the help of equivalent circuits using ZSimpWin (Princeton Applied Research) and the data were presented in Nyquist plots. Importantly, all measurements were repeated for a minimum of three times with separate electrodes to obtain statistically meaningful results.

### X-Ray Photoelectron Spectrometry (XPS)

XPS spectra were employed to characterize the formed Fc-modified lipoic acid derivative film on the sputtering gold electrode surface. The electrode surface was cleaned with 1 M H₂SO₄ for 5 min, then washed with Millipore water and sonicated in ethanol for 10 min to reduce the formed gold oxide. After drying with N₂, the electrode was incubated with 2 mM of the Fc-modified lipoic acid derivative for 24 h at room temperature. Prior to XPS experiments, the electrode surface was thoroughly washed with ethanol and dried with N₂. The XPS spectra were acquired with a Kratos Axis Ultra spectrometer (Kratos Analytical, UK) using a monochromatic Al-Kα X-ray source (15 mA, 14 kV). The takeoff angle between the film surface and the photoelectron energy analyzer was 90°. A typical operating pressure was around 5×10⁻¹⁰ Torr in the analysis chamber. Survey spectra (0-1100 eV) were taken at constant analyzer pass energy of 160 eV and were applied on an analysis area of 300×700 μm. High-resolution analyses were carried out at a pass energy of 20 eV on the same surface area. The binding energies were referenced to Au 4f_{7/2} at 83.96 eV and the spectrometer dispersion was adjusted to give a binding energy of 932.62 eV for the Cu 2p_{3/2} line of metallic copper. Acquired spectra were charge-corrected to the main line of the carbon 1s spectrum (adventitious carbon) set at 284.8 eV and analyzed using CasaXPS software (version 2.3.14).

### Time-of-Flight Secondary Ion Mass Spectrometry (TOF-SIMS)

Sputtering gold electrodes, prepared under the same protocol taken in the XPS experiments, were used for TOF-SIMS analyses. TOF-SIMS experiments were performed using TOF-SIMS IV (ION-TOF GmbH, Munster, Germany) which was equipped with a Bi liquid metal ion source. For all measurements, a 25 keV Bi³⁺ cluster primary ion beam with a pulse width of 12 ns (target current of −1 pA) was utilized. The cycle time for the bombardment processes and detection was 100 μs (or 10 kHz). A pulsed, low energy electron flood was used to neutralize sample charging. For each sample, spectra were collected from 128×128 pixels over an area of 500 μm×500 μm for 60 s. Positive and negative ion spectra were internally calibrated by using H⁺, H²⁺, CH³⁺, H⁻, C⁻, and CH⁻ signals, respectively. Two spots per sample were analyzed by using a random approach.

### Results

Binding of the HIV RT enzyme was electrochemically detected in each case, using either the labelled or unlabeled electrode.

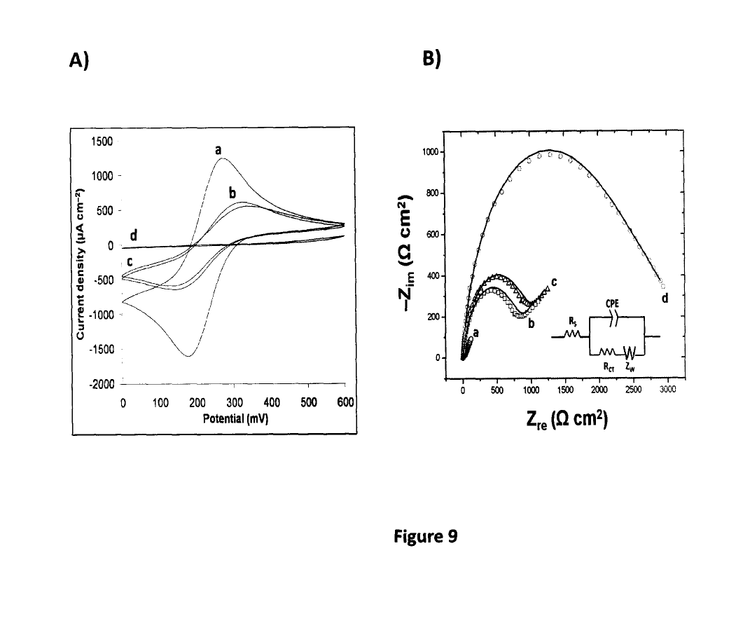

FIG. 9 illustrates the cyclic voltammograms (A) and Faradic impedance spectra (B) of the unlabeled probe or biosensor after each immobilization or binding step in 10 mM sodium phosphate buffer (pH 7), containing 5 mM NaClO₄ and 5 mM K₄Fe(CN)₆. (a) bare gold; (b) after coating with lipoic acid NHS ester; (c) after covalent binding of the RT-specific peptide; (d) after back-filling with 10 mM hexanethiol. The cyclic voltammograms were recorded at a scan rate of 100 mV s⁻¹. The impedance spectra were recorded from 100 kHz to 0.1 Hz and the amplitude was 0.25 V vs Ag/AgCl. Measured data are shown as symbols with the fitting to the equivalent circuit as solid lines. The inset shows the equivalent circuit applied to fit the measured impedance data as, R_{S} solution resistance; CPE constant phase element, R_{CT} charge transfer resistance, and Z_{W} is the finite length Warburg impedance.

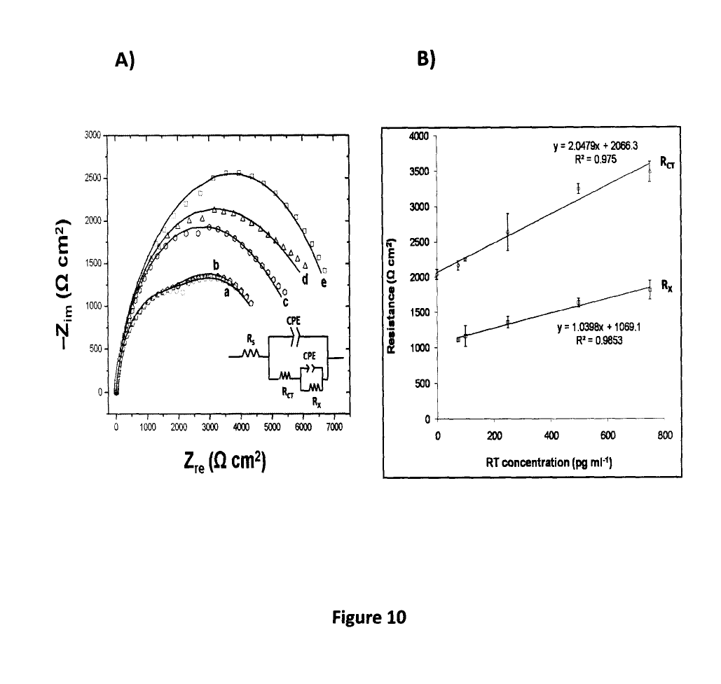

The unlabeled probe was incubated with increasing RT concentrations as follows: (a) 75; (b) 100; (c) 250; (d) 500; (e) 750 pg ml⁻¹. Impedance measurements were performed at a formal potential of 0.25 V vs Ag/AgCl in 10 mM sodium phosphate buffer (pH 7), containing 5 mM NaClO₄ and 5 mM K₄Fe(CN)₆. The frequency was from 100 kHz to 0.1 Hz. A Nyquist plot (−Zᵢₘ vs Zᵣₑ) of impedance spectra obtained at different RT concentrations is shown in FIG. 10A. Measured data are shown as symbols with the fitting to the equivalent circuit as solid lines. The inset shows the equivalent circuit applied to fit the measured impedance data as, R_{S} solution resistance; CPE constant phase element, R_{CT} charge transfer resistance, and R_{X} is the RT resistance. Equivalent circuit element values for the lipoic acid NHS ester-modified gold electrode, covalently coupled to RT-specific peptide, in the presence of increasing concentrations of RT are set out in Table 2. FIG. 10B is calibration plot of R_{CT} and R_{X} vs RT concentration. Error bars represent the standard deviation of triplicate measurements (n=3).

TABLE 2

| RT conc. | R_{S} | CPE | | R_{CT} | CPE | | R_{X} |
|---|---|---|---|---|---|---|---|
| (Pg ml⁻¹) | (Ω cm²) | (μF cm²) | n | (Ω cm²) | (μF cm²) | n | (Ω cm²) |
| 0 | 2.3(0.01) | 38.2(0.8) | 0.9(0.01) | 2040(70.4) | | | |
| 75 | 2.3(0.01) | 38.4(0.8) | 0.9(0.01) | 2168(64.7) | 87.6(4.3) | 0.4(0.01) | 1118(27.7) |
| 100 | 2.2(0.01) | 37.5(0.2) | 0.9(0.01) | 2251(24.2) | 74.8(9.4) | 0.4(0.01) | 1164(145.5) |
| 250 | 2.3((0.02) | 32.2(2.9) | 0.9(0.01) | 2635(257.5) | 66.7(3.9) | 0.4(0.01) | 1356(79.7) |
| 500 | 2.3(0.01) | 26.3(0.8) | 0.9(0.01) | 3248(71.6) | 61.2(2.4) | 0.4(0.01) | 1637(63.5) |
| 750 | 2.3(0.01) | 23.6(1.6) | 0.9(0.01) | 3486(144.3) | 59.4(4.4) | 0.4(0.01) | 1812(132.8) |

^{a}The values in parentheses represent the standard deviations from at least three electrode measurements.

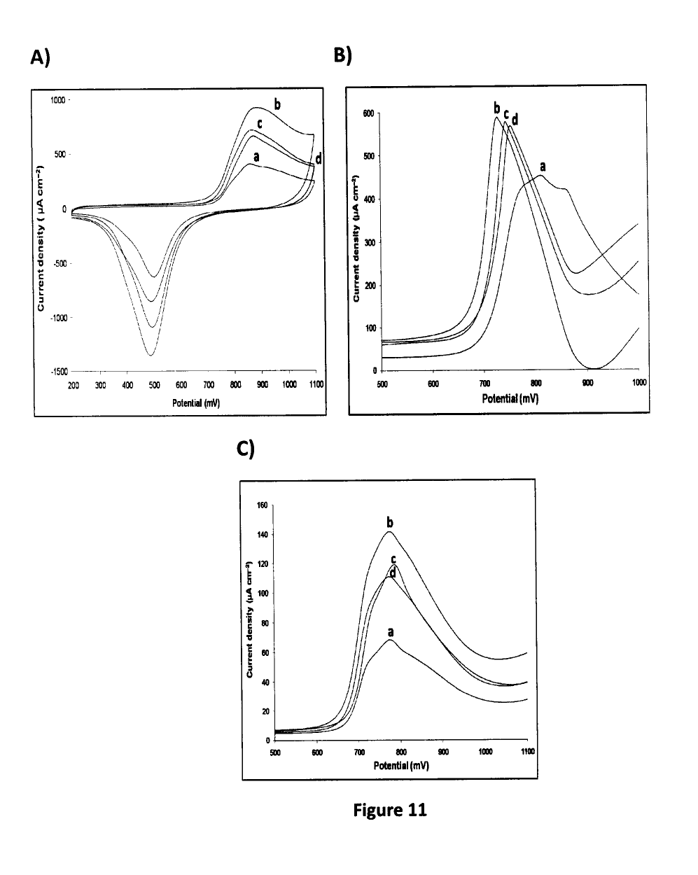

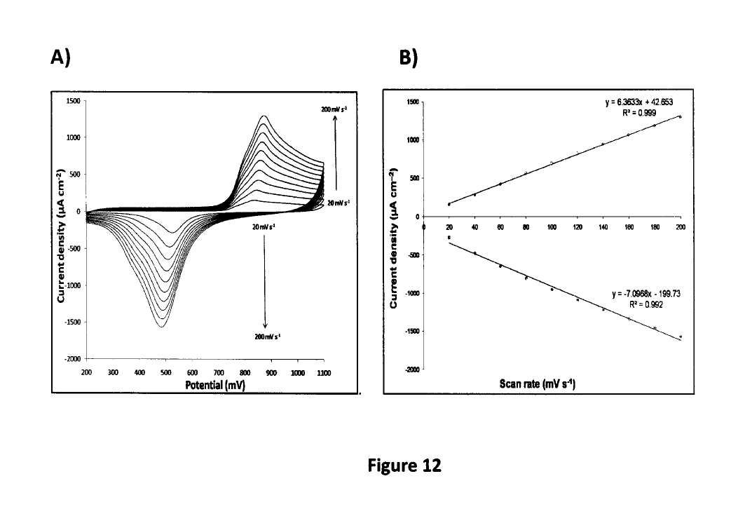

FIG. 11 illustrates cyclic voltammograms (A), square wave voltammograms (B) and differential pulse voltammograms (C) of the labeled RT probe or biosensor after each immobilization or binding step in 10 mM sodium phosphate buffer (pH 7). (a) bare gold; (b) after coating with lipoic acid NHS ester; (c) after covalent binding of the RT-specific peptide; (d) after back-filling with 10 mM hexanethiol. The cyclic voltammograms were recorded at a scan rate of 100 mV s⁻¹ vs Ag/AgCl. FIG. 12 illustrates cyclic voltammograms (A) of the Fc-labeled lipoic acid modified gold electrode in 10 mM sodium phosphate buffer (pH 7) at different scan rates ranging from 20 to 200 mV s⁻¹. The linear relationship between the scan rate and the anodic and cathodic peak currents for the bound film is illustrated in FIG. 12B. Error bars represent the standard deviation of triplicate measurements (n=3).

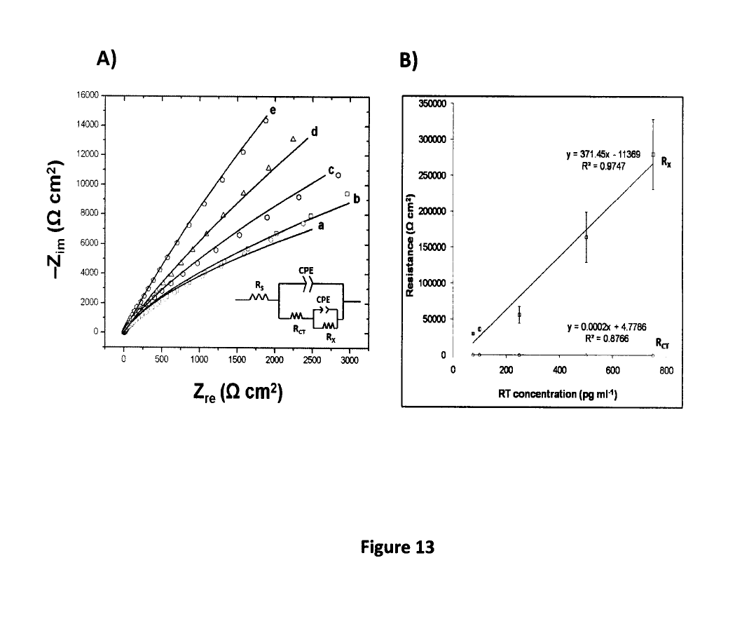

A Nyquist plot (−Zᵢₘ vs Zᵣₑ) of impedance spectra obtained at different RT concentrations: (a) 75; (b) 100; (c) 250; (d) 500; (e) 750 pg ml⁻¹ using the labelled probe is shown in FIG. 13(A). Impedance measurements were performed at a formal potential of 0.75 V vs Ag/AgCl in 10 mM sodium phosphate buffer (pH 7). The frequency was from 100 kHz to 0.1 Hz. Measured data are shown as symbols with the fitting to the equivalent circuit as solid lines.

The inset shows the equivalent circuit applied to fit the measured impedance data as, R_{S} solution resistance; CPE constant phase element, R_{CT} charge transfer resistance, and R_{X} is the RT resistance. A calibration plot of R_{CT} and R_{X} vs RT concentration is shown in FIG. 13B. Error bars represent the standard deviation of triplicate measurements (n=3). Equivalent circuit element values for the Fc-labeled lipoic acid-modified gold electrode, covalently coupled to RT-specific peptide, in the presence of increasing concentrations of RT are set out in Table 3.

TABLE 3

| RT conc. | R_{S} | CPE | | R_{CT} | CPE | | R_{X} |
|---|---|---|---|---|---|---|---|
| (Pg ml⁻¹) | (Ω cm²) | (μF cm²) | n | (Ω cm²) | (μF cm²) | n | (Ω cm²) |
| 75 | 0.002(0.04) | 0.6(0.07) | 0.9(0.02) | 4.79(0.03) | 156.9(6.7) | 0.9(0.02) | 29460 |
| | | | | | | | (1322.4) |
| 100 | 297.1 × 10⁻⁶ | 0.5(0.08) | 1(0.02) | 4.81(0.06) | 123.3(13.7) | 0.9(0.02) | 35810 |
| | (0.04) | | | | | | (2713.5) |
| 250 | 8.4 × 10⁻⁵ | 0.7(0.06 | 0.9(0.01) | 4.83(0.02) | 108.2(8.5) | 0.9(0.01) | 55970 |
| | (2.2 × 10⁻⁶) | | | | | | (11685.6) |
| 500 | 2.1 × 10⁻⁶ | 0.6(0.01) | 0.9(0.01) | 4.84(0.001) | 93.7(1.7) | 0.9(0.01) | 164300 |
| | (1 × 10⁻⁶) | | | | | | (34958.6) |
| 750 | 9.4 × 10⁻⁷ | 0.7(0.02) | 0.9(0.01) | 4.94(0.01) | 86.7(1.8) | 0.9(0.01) | 279800 |
| | (7 × 10⁻⁸) | | | | | | (48901.6) |

^{a}The values in parentheses represent the standard deviations from at least three electrode measurements.

1. A redox probe unit comprising a redox probe modified to incorporate a detector-binding moiety adapted to bind a detector having the capacity to specifically interact with a target.

2. (canceled)

3. The redox probe unit as defined in claim 1, wherein the redox probe comprises an electrode linked to a molecule that undergoes a reversible one-electron redox process.

4. The redox probe unit as defined in claim 3, wherein the molecule is selected from the group consisting of a metallocene, quinones, anthraquinone, [Ru(NH3)6]2+/3+ and [Ru(bipy)3]2+/3+.

5. The redox probe unit as defined in claim 4, wherein the metallocene is selected from the group consisting of ferrocene, cobaltocene, and derivatives thereof.

6. The redox probe unit as defined in claim 1, wherein the detector-binding moiety is suitable to form a linkage with a peptide, protein or oligonucleotide.

7. The redox probe unit as defined in claim 6, wherein the detector-binding moiety is selected from the group consisting of carboxyl, amine, hydroxide, sulfhydryl, active ester and halide.

8. An electroactive biodetector comprising a redox probe modified to incorporate a detector-binding moiety to which is bound a detector having the capacity to interact with a target.

9. (canceled)

10. The biodetector defined in claim 8, wherein the redox probe comprises an electrode linked to a molecule that undergoes a reversible one-electron redox process.

11. The biodetector defined in claim 10, wherein the molecule is selected from the group consisting of a metallocene, quinones, anthraquinone, [Ru(NH3)6]2+/3+ and [Ru(bipy)3]2+/3+.

12. The biodetector defined in claim 11, wherein the metallocene is selected from the group consisting of ferrocene, cobaltocene, and derivatives thereof.

13. (canceled)

14. The biodetector as defined in claim 8, wherein the detector-binding moiety is selected from the group consisting of carboxyl, amine, hydroxide, sulfhydryl, active ester and halide.

15. The biodetector as defined in claim 8, wherein detector is a peptide or nucleic acid ligand.

16. The biodetector as defined in claim 15, wherein the detector is a ligand for a viral protein.

17. The biodetector as defined in claim 16, wherein the viral protein is an HIV-1 enzyme selected from the group consisting of HIV-1 reverse transcriptase, HIV-1 integrase and HIV-1 protease.

18. The biodetector as defined in claim 15, wherein the detector is a ligand for a non-viral protein selected from the group consisting of antibodies, hormones and antigens.

19. A method of preparing an electroactive biodetector as defined in claim 8 suitable for the detection of a target comprising the steps of:
i) modifying a redox probe to incorporate a detector-binding moiety on the surface thereof; and
ii) attaching to the detector-binding moiety a detector which has the capacity to specifically interact with the target.

20. An electrochemical method of detecting a target in a sample comprising the steps of:
i) exposing the sample to a redox probe modified to include a detector that is suitable to bind to the target, wherein the detector is linked to the probe via a detector binding moiety, and
ii) measuring the electrochemical signal produced by the redox probe, wherein a change in the electrochemical signal of the probe as compared with a control signal is indicative of the presence of the target in the sample.

21-30. (canceled)

31. The method of claim 20, wherein the signal is measured by one of cyclic voltammetry, differential pulse voltammetry, square wave voltammetry, alternating current voltammetry and impedance spectroscopy.

32. (canceled)

33. An electroactive biodetector unit adapted to detect multiple targets, wherein the biodetector unit comprises multiple biodetectors as defined in claim 8, wherein each of said biodetectors is adapted to detect a different target.

34. The biodetector unit of claim 33, wherein each biodetector comprises a redox probe modified to incorporate a detector-binding moiety to which is bound a detector and each detector is adapted to detect a different targets.

35. The biodetector unit of claim 34, wherein each detector is adapted to detect a different HIV-1 protein selected from the group consisting of HIV-1 reverse transcriptase, HIV-1 integrase and HIV-1 protease.
